(19) United States
(12) Patent Application Publication
(10) Pub. No.: US 2025/0241613 A1
(43) Pub. Date: Jul. 31, 2025

(54) ASTHMA DIAGNOSIS AND MANAGEMENT SYSTEM

(71) Applicant: Trudell Medical International Inc., London (CA)

(72) Inventors: Sean Alexander Marshall, London (CA); Stephen Costella, London (CA); Britton Sprickerhoff, London (CA); Mark Nagel, Mt. Brydes (CA); Jackson Yu, Toronto (CA)

(73) Assignee: Trudell Medical International Inc., London (CA)

(21) Appl. No.: 19/036,586

(22) Filed: Jan. 24, 2025

Related U.S. Application Data
(60) Provisional application No. 63/625,176, filed on Jan. 25, 2024.

(51) Int. Cl. A61B 7/04; A61M 15/00

(52) U.S. Cl. CPC A61B 7/04; A61M 15/0001; A61B 2503/06; A61M 2205/3375

(57) ABSTRACT

An asthma diagnosis and management system including a valved holding chamber defining an interior cavity, a backpiece having an opening configured to receive a pressurized metered dose inhaler and a user interface. A microphone is coupled to the valved holding chamber and is in communication with an interior of the valved holding chamber and is configured to capture a sound of a user's lungs.

This application claims the benefit of U.S. Provisional Application No. 63/625,176, filed Jan. 25, 2024 and entitled “Asthma Diagnosis and Management System,” the entire disclosure of which is hereby incorporated herein by reference.

### BACKGROUND

Asthma is a common condition from birth that affects the airways of the lungs, causing them to narrow and produce extra mucous. This condition often results in difficulty breathing, coughing, wheezing, and/or shortness of breath during periods with increased physical activity or even during everyday activities. It is important to initiate the treatment of asthma early to reduce the physical, social and emotion burden of disease on both the individual, family members, and/or friends, yet early treatment requires the disease to be accurately diagnosed by a trained physician.

The current standard in diagnosing asthma is taking a spirometry test. This is a common lung function test used to diagnose and monitor asthma. This test involves forcefully exhaling into a device that measures the volume of air and the speed of exhalation and is used to determine if there is any obstruction or limitation in the airways. These results are compared against averages for age, sex, ethnicity, height, and other factors, and results below a set threshold quantitively indicate the individual may have asthma. Physicians often use spirometry as an objective measure of airflow limitation, in conjunction with subjective clinical assessment, like asking about symptoms and medical history and conducting a physical examination, to make a diagnosis of asthma.

Children under the age of 6 (i.e. preschool children) generally do not have the coordination, understanding, or capacity to undergo such maneuvers nor is there sufficient reference data for this population to inform whether obtained values are clinically significant. As such, spirometry may not be effectively performed in children under the age of 6. Currently there is no widely accepted objective test to identify asthma in preschool children.

Compounding the problems with asthma diagnosis is that the symptoms of wheezing and breathlessness may occur in up to two thirds of preschool children and are a leading cause of ER visits among the age group. These symptoms are not always a direct result of asthma, as asthma and other conditions share significant overlap in symptoms that make it difficult to diagnose. Additionally, these overlapping symptoms manifest differently from child to child, and even manifest differently from season to season within the same child making it difficult to make a sound diagnosis. Although wheezing and breathlessness are typically associated with viral respiratory infections, the lack of accurate spirometry results makes it difficult to ascertain when the symptoms are caused by asthma. As recurrent preschool wheezing can be associated with substantial morbidity and impact long term health, there is a motivation to expeditiously make a correct diagnosis and treat the condition. Furthermore, asthma medications have known side effects and have associated cost, so it is also beneficial to avoid overdiagnosis.

This difficulty in identifying asthma in preschool aged children generally results in high costs to the healthcare system as concerned parents and their children are constantly shuffled between doctors and bounced around the healthcare system while looking for a sound diagnosis. These frequent visits and endless tests may also be traumatic for the young child who do not understand what is going on.

The guideline supported diagnostic approach for preschool children's asthma rules is diagnosis via risk factors and history taking and evaluating the patient's response to a therapeutic trial of asthma medication. However, currently evaluating a child's response to medication requires relying on subjective parental accounts.

For a therapeutic trial, the physician prescribes asthma medication to a child for a period of time (e.g. 3 months) and compares the condition of the child before medication, to when the child is on medication. These primary observations from the doctor are limited as there is no guarantee that the child will present any asthma symptoms with either the initial visit or during the follow-up visit. Additionally, there is no way for the physician to know firsthand the extent in which the child complied to the prescribed medication regime and/or whether the child took their medications properly.

It is up to the physician to interpret the outcomes of the therapeutic trial through the subjective account of the parent or guardian. Through these subjective accounts the physician must decipher several indicators, including medication adherence, long term trends in symptoms, change in symptoms pre/post reliever, triggers, frequency of reliever use, frequency of illness, trends in activity level, and trends in nocturnal cough, among other indicators. It is important to emphasize the subjective nature of a parental account as some parents may down-play or overemphasize the outcomes of a therapeutic trial to sway the physician into making a biased diagnosis. This adds another layer of complexity and uncertainty onto an already difficult to diagnose disease.

As a result of this difficulty, some physicians may wait to officially diagnose a child with suspected asthma until they are of the age where spirometry can be performed. Although this is beneficial in preventing misdiagnosis, if the child has a respiratory disease that goes undiagnosed the child could miss out on essential treatment for what can become a span of years. This can also be difficult on the parent as they often experience significant anxiety related to their child's asthma diagnosis, especially during periods of frequent exacerbations. In other extremes, physicians may liberally prescribe medication to children who do not have asthma. Such practices expose children to side-effect-causing medication that in no way helps manage the disease.

Previous attempts have been made to resolve the issue of diagnosing pediatric asthma cases. For example, wheeze monitors, electronic stethoscopes, and/or wearable respiration monitors typically require integrating an additional device into an asthma patient's management routine. Adding a device requires the patient or caregiver to understand how to use the device, understand when to use the device, carry the device on their person, and/or have the motivation or time to use the device during an exacerbation. There are often additional costs associated with this as well, either to the patient/caregiver or the healthcare system in the case where the technology is reimbursed. When the user is having an exacerbation, often their thoughts and actions will be centered on activities that relieve the exacerbation, rather than capturing information to be used for managing their disease down the line. There is also an inherent trend of poor adherence to medication when one's asthma has temporarily improved. In these situations, an additional device may be forgotten or omitted from use because the user does not feel it is necessary.

Similarly, other solutions typically require conscious effort to take measurements from the patient. This may include for example performing auscultation using a stethoscope and/or manual logging method via an app. For example, if a user sees no direct correlation to an improvement in symptoms through using such a device, the user may not deem the effort of using the devices and/or logging the information worthwhile and may stop using the device altogether. In other examples, the devices focus on a narrow range of measures that are used to indicate asthma in a patient. While there is inherently nothing wrong with this approach, these solutions may miss a range of other asthma indicators which could assist in a quicker diagnosis. If additional measures are to be recorded, additional devices would be needed, which feeds back into the outlined problem of implementing new or too many devices to diagnose or manage asthma.

Additionally, systems may track when medication was administered, but those systems in the pediatric asthma space typically do not provide any other information about the quality of the medication administration. As a result, vital information may be missing when physicians interpret the data as they have no way to tell the quantity of drug that was administered to their patients' lungs. For example, a patient may repeatedly administer the medication but have no medication reach the lungs, which may result in an inaccurate diagnosis from the physician.

Finally, some solutions are limited to the management of asthma symptoms, which does not assist in the diagnosis of pediatric asthma.

For these various reasons, it is apparent that no singular system is available for supporting physicians in making a diagnosis for preschool patients while taking into consideration the many symptoms related to the disease and also being easy to use.

### BRIEF SUMMARY

Due to the various issues regarding pediatric asthma, there is a need to provide quantitative and objective information of value to agents that make decisions through the pediatric patient journey. Doing so will allow for a quicker and a more accurate diagnosis of pediatric asthma, with less costs incurred to the medical system. In one embodiment, a system incorporates a solution into the user's current regime, thus minimizing the addition of any additional steps and devices and removing potential barriers to continued use.

In one aspect, one embodiment of an asthma diagnosis and management system includes a valved holding chamber defining an interior cavity, a backpiece having an opening configured to receive a pressurized metered dose inhaler and a user interface. A microphone is coupled to the VHC and is in communication with an interior of the valved holding chamber and is configured to capture a sound of a user's lungs.

### BRIEF DESCRIPTION OF THE DRAWINGS

FIG. 1 is a schematic representation of one embodiment of an asthma diagnosis and management system.

FIG. 2 is a perspective view of one embodiment of a smart valved holding chamber in communication with parent/patient facing application.

FIG. 3 is a perspective view of one embodiment of a smart valved holding chamber.

FIG. 4 is an end view of a portion of a smart valved holding chamber with a microphone positioned in an in an MDI adapter backpiece.

FIG. 5 shows a schematic for a microphone and supporting hardware architecture.

FIG. 6 discloses other embodiments of a lung sound breath recording element for the smart valved holding chamber.

FIG. 7 shows another smart valved holding chamber embodiment.

FIG. 8 shows another smart valve holding chamber embodiment and ambient sound monitoring device.

FIG. 9 discloses one embodiment of a bedside placement of the smart valved holding chamber and ambient sound monitoring device.

FIG. 10 discloses one embodiment of a system including an ambient sound monitoring device embodiment, wireless connection, lighting, and display.

FIG. 11 discloses one embodiment of a system including an ambient sound monitoring device embodiment, cable connections and locking mechanisms.

FIG. 12 discloses an ambient sound monitoring device embodiment, integrated into the smart VHC.

FIG. 13 illustrates one embodiment of a physician facing dashboard/application.

### DETAILED DESCRIPTION OF THE DRAWINGS AND THE PRESENTLY PREFERRED EMBODIMENTS

It should be understood that the term “plurality,” as used herein, means two or more. The term “longitudinal,” as used herein, means of or relating to a length or lengthwise direction, for example a direction running from a back to a front of a valved holding chamber (VHC). The term “lateral,” as used herein, means situated on, directed toward or running in a side-to-side direction VHC. The term “coupled” means connected to or engaged with whether directly or indirectly, for example with an intervening member, and does not require the engagement to be fixed or permanent, although it may be fixed or permanent. The terms “first,” “second,” and so on, as used herein, are not meant to be assigned to a particular component or feature so designated, but rather are simply referring to such components and features in the numerical order as addressed, meaning that a component or feature designated as “first” may later be a “second” such component or feature, depending on the order in which it is referred. It should also be understood that designation of “first” and “second” does not necessarily mean that the two components, features or values so designated are different, meaning for example a first direction may be the same as a second direction, with each simply being applicable to different components or features.

The present disclosure relates to a system to aid a physician in diagnosing asthma, including preschool asthma, or other asthma cases with or without spirometry, according to the physician's judgement. This system aims to gather quantitative and objective information passively that cannot be observed during short visits to the physician's office, for example due to the episodic nature of asthma.

In one embodiment, the system includes a smart valved holding chamber (VHC) (e.g., as disclosed for example and without limitation in U.S. Pat. No. 10,850,050 B2, and U.S. Pat. No. 11,395,890 B2, the entirety of which are hereby incorporated herein by reference). The various elements of the current embodiments are features, attributes, and/or additional devices that are designed to integrate into the use of such a VHC. One exemplary embodiment of a smart VHC includes a chamber housing 100 having a wall defining an interior space extending along a longitudinal axis/inhalation flow path, a back piece 106 coupled to an input end of the chamber housing and a mouthpiece and/or valve assembly 112 coupled to an output end of the chamber housing 102. The valve assembly 112 defines a second chamber upstream of the chamber defined by the housing 102. The mouthpiece assembly may be releasably and removably coupled to the chamber housing, for example with tabs received in grooves. The mouthpiece and/or mask 15 may be configured with an inhalation valve and/or an exhalation valve, which provides an inhalation and exhalation flow path respectively. The inhalation and exhalation valves may alternatively be disposed on other components of the VHC. In various embodiments, a valve is configured as part of an annular donut valve, having an inner periphery that defines the inhalation valve and an outer periphery defining an exhalation valve. In other embodiments, the inhalation valve is configured as a duckbill valve, which may also have an outer annular flange defining the exhalation valve. In other embodiments, the inhalation and exhalation valves may not be integral, but rather are separately formed and disposed within the VHC. The backpiece is configured with an opening, which is shaped to receive a mouthpiece portion of a pressurized metered dose inhale (pMDI) 125, which includes an actuation boot and medicament container. The boot further includes a chimney portion defining a cavity shaped to receive a medicament container. The boot further includes a support block defining a well shaped to receive a valve stem of the MDI. The well communicates with an orifice, which releases aerosolized medication into the interior space of the chamber housing. Various embodiments of the VHC and MDI, including the mouthpiece assembly, chamber housing and backpiece, are disclosed for example and without limitation in U.S. Pat. Nos. 6,557,549, 7,201,165, 7,360,537 and 8,550,067, all assigned to Trudell Medical International, the Assignee of the present application, with the entire disclosures of the noted patents being hereby incorporated herein by reference.

### Medication Adherence and Technique Monitoring

In the present disclosure, medication adherence instances may be collected to determine the overall adherence to the prescribed therapeutic trial. In combination with medication adherence, the device will have a function to calculate the technique of the user while taking the medication. The technique is calculated through an analysis and combination of the available metrics that were recorded around the time of the medication delivery. The data and metrics may be used to gain an understanding of the respirable dose received by the user.

These combined features convey useful information to physicians that assists in a pediatric asthma diagnosis, as it outlines the overall adherence and quantity of medication that reached the lung during the therapeutic trial. This objective account will remove the need for the physician to decipher the information conveyed by a parent during a visit. This element will also enable physicians to identify individuals who struggle while taking their medication and provide an opportunity for correcting patient behaviors.

To collect this information, sensors may be placed within the same device that collects medication adherence to understand essential metrics in inhaler technique. These metrics may include at least one of: body position, inhalation speed, volume of inhalation, inhaler identification, inhaler actuation timing, flow rate through the device, orientation of the device, or other metrics not mentioned.

### Ambient Sound Monitoring

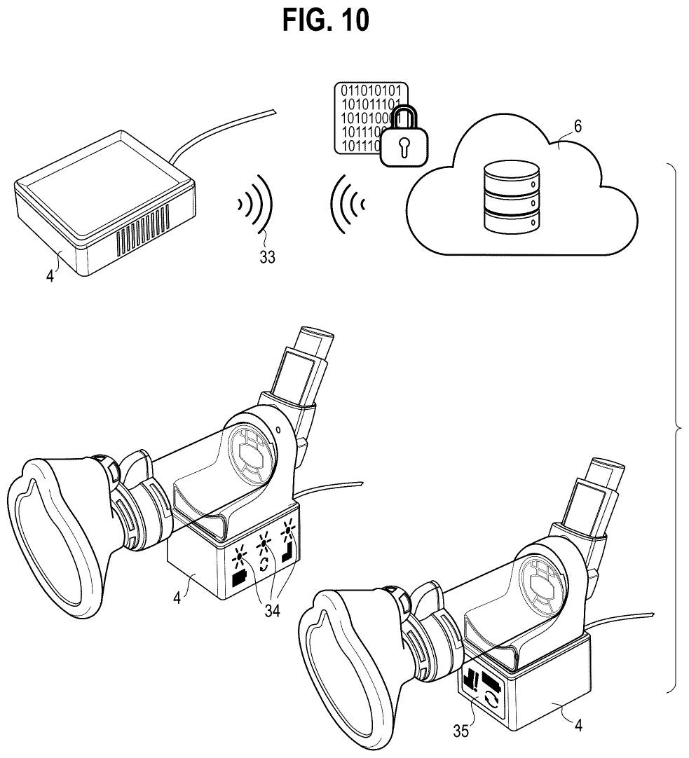

Another element of the present disclosure may include the ability for the system to monitor and track breathing sounds, for example coughing sounds, in patients over long periods of time when the patient is in the same vicinity as the device. One example of this application would be to track ambient sounds, such as the coughs of a preschool asthma patient throughout the duration of the night, as shown for example in FIG. 9.

This information will be captured through a device equipped with a microphone 18 This device may be the smart valved holding chamber, or an accessory device to the smart valved holding chamber. This device may either be manually or automatically triggered to listen to the environment for identifiable respiratory noises, or other ambient sounds. Through artificial intelligence, or other preprogrammed software, the recorded sounds may be analyzed for important data that may be conveyed to caregivers, patients, or physicians. This analysis may take place on the smart valved holding chamber, a smart valved holding chamber accessory, a companion mobile app or a server. This data includes at least one of the following measures: frequency of coughs, severity of cough, total number of coughs, identifying characteristics and trends of each cough, classification of each cough, etc. The processed information will be transmitted to the patient or physician's information portal where it may be displayed for interpretation by the user.

The device, in particular the smart valved holding chamber 3, is designed so that it does not introduce any new adherence steps beyond initialization. Once initialization is complete, the device, e.g., the valved holding chamber 3, may be positioned close to the child when sleeping (e.g., the bedside). Proximity to the child may also enable the smart valved holding chamber 3 to be easily accessed in times of an exacerbation.

### Lung Sound Breathing Recording

Similarly, another element of the present disclosure may be the ability for the smart valved holding chamber 3 to listen to breathing sounds generated when the patient takes medication through the smart valved holding chamber 3. This sound recording is of much higher fidelity than the ambient sound monitoring feature as the microphone 18 will be positioned at roughly the same distance from the lungs (minus any anatomical variation), in the same position at the user's mouth, and without any major barriers that muffles or distorts clinically important sound artifacts. This higher fidelity sound will be analyzed and compared to gather insights regarding the patient's respiratory health.

The lung sound breath recording feature is completely passive and begins recording audio data generated from the lungs when the patient utilizes the Smart Valved Holding Chamber to administer medication from a pressurized metered dose inhaler (pMDI). This action is passive as it ties the act of using a valved holding chamber with sound recording, ensuring that data will be collected every time the user administers medication using the Smart Valved Holding Chamber device. Children are typically use a Valved Holding Chamber when taking medication from a pMDI as they do not have the coordination necessary to inhale when the drug is actuated. This further enforces the passive nature lung sound breath recording when using a Smart Valved Holding Chamber.

In addition, a rescue medication is generally only taken when an individual needs a rapid relief of symptoms, indicating that they are having some form of breathing abnormalities. By coupling the lung sound breath recording feature set with the rescue medication delivery, it ensures that the exacerbated breathing will be captured.

In other uses such as inhaling with an Inhaled corticosteroids (ICS, the device may alert the parent in some way if the child's breath sounds indicate some form of wheeze, cough, congestion, or other difficulty breathing, and suggest that the parent take action in an appropriate manner. There is also a feature that enables breath sound recordings a period of time after rescue medication was administered. At that time, the user will be prompted to breathe through the device again without dispensing medication. In this second time the user will breathe through the device as normal, but they will not administer any medication. During this time, the smart valved holding chamber will record any breath sounds. The purpose of this post-reliever audio capture is to be able to discern differences between the two audio recordings and evaluate the user's response to the initially administered rescue medication. Of particular interest is the resolution of wheeze sounds following rescue medication. Any change in symptoms between before and after reliever medication use may be a strong indication that an individual has asthma.

Like the last example, the user may also use the device without medication to listen for abnormal respiratory sounds, or to record other breathing parameters like flow through the device without being prompted. The device would then analyze the results and provide insights to the user's respiratory condition with instructions on how to proceed. For example, after breathing through the Smart Valved Holding Chamber the device may validate that the child has a wheeze and instruct the parent and child to administer a rescue medication, or other applicable action. While this added feature introduces a new step the child is not fully familiar with, it is on a device that they are already acquainted with using during drug delivery, so the barriers to adherence are significantly reduced.

A further explanation of the evaluation may be found in the “Algorithmic Interpretation of Breathing Sounds” section below.

The Lung Sound Audio Recording element improves upon prior medication management systems as it leverages the widespread knowledge and current practice of using valved holding chambers to gather information that can help inform physicians on proper diagnosis.

It has historically been a challenge for physicians to convince their patients to adhere to the guidance given to them, whether it is medication that needs to be taken, information that needs to be logged, exercises that need to be performed, etc. Adherence to respiratory related medical conditions is no different, and adherence in this space tends to be below average. Adherence is a multifaceted problem and is affected by numerous factors including, motivation, knowledge of how to use the prescribed medication/device, time to complete the action, etc.

### Algorithmic Interpretation of Breathing Sounds

The algorithm could be a smart learning algorithm, or an artificial intelligence (AI) that is used to sort through the data that is collected. This collected data includes breathing sounds (both ambient and direct from lungs), technique, adherence data, airflow information, or other data stored on, or recovered from one or multiple devices that were mentioned in this disclosure. From this information, the artificial intelligence may make recommendations based upon one, or a combination of multiple data points that it will present to parents or physicians. The Al may even make recommendations on next actions to take, or the proper diagnosis of the patient.

The AI may also create a sound profile based upon inputs for a specific child (as respiratory sounds are different between children). In doing so, the Al would learn the specific respiratory sounds of the child and better identify different sounds.

The device may accomplish most of the data manipulation or learning either on the cloud, on the phone, or on one of the devices.

In one embodiment, data transfers may be made between from the device to the cloud for processing. Physicians and users may access a portal to review and analyze the presented information.

### Information Portals

The information portals are applications that function to portray the collected data to interested parties. There may be multiple portals, including a physician facing portal, and a parent/guardian/caregiver facing portal. In both portals, all the data collected from the aforementioned elements are displayed in a concise and distinct manner to enable parents and physicians to make educated decisions about the outcome of the therapeutic trial, diagnosis, and the overall management of the patient's condition. The patient/caregiver facing application may also reduce anxiety and improve engagement in the management of the disease.

The parent facing application may permit a parent, or other caregiver, to enter symptoms, triggers, past medical history, or other questions that the app would prompt that would be useful for the physician to make a diagnosis. This portal also has a feature set that allows the parent/guardian/caregiver to share pertinent information regarding the entered and collected data to the physician using a PDF, or other suitable electronic medium. This method would include means to share pertinent information regarding the therapeutic trial, or other information about the child's condition, including at least one of but not limited to: audio recordings, adherence data, technique information, or any algorithmic or Al interpretation of the collected data. When the app is connected to the internet, it will push the information to a cloud storage where the information can be readily viewed by the physician through a physician facing dashboard.

The physician facing portal would share similar feature sets to the parent portal, but it may include other pertinent information, like differential diagnoses, family history, and other necessary information. This portal also would have an ability to be fully integrated with major electronic medical records (EMRs), which would enable the physician to store the resulting information in a method that integrates with their workflows.

### System

In one embodiment, the system couples the act of taking medication with a valved holding chamber to recording clinical quality data that can be used in the management and diagnosis of respiratory conditions. Additionally, the system, including the valved holding chamber, has the functionality of recording sounds generated from within the lungs, or from the ambient environment. The system acts as a tool that physicians can use to gather information to review prior to making a diagnosis, and as a post-diagnosis management tool to ensure that patients, especially pediatric patients, are living to their fullest potential. In this system, the information generated and presented to a physician encompasses a wide breadth of insights that may be difficult for physicians to determine without the use of this tool. This information includes the data such as wheeze detection, cough monitoring, symptom tracking through the included app and medication and technique tracking. All this information is presented on the patient and physician facing app/dashboard with included algorithmic interpretations on some of the data, like respiratory sound identification.

Moreover, the embodiments of the system includes tools used to gather information about the pediatric patient using widely adopted practices, such as the use of a valved holding chamber, or are completely passive and require no additional thought to record information beyond the initial set up of the device, like the Ambient Sound Monitoring Device. Ultimately, the combination of passive data that covers a wide range of parameters, used in the diagnosis of pediatric asthma saves all parties involved a significant amount of money, time, and uncertainty. This is because the system works to provide passively collected information to physicians which enables them to make educated diagnoses on children with respiratory symptoms without the use of spirometry. Additionally, after diagnosis, the system empowers parents and caregivers to take charge of their child's condition. These factors work to reduce time spent within, and the attendant strain on, the healthcare system.

In the present disclosure, the embodiments of the devices and systems may be used to objectively capture concrete information to either enable a faster diagnosis of respiratory conditions, or better track and manage asthma. If used alongside a physician for diagnosis purposes, the use of the system may be used as part of a therapeutic trial. The system is to be referred by a physician, but a parent/caregiver or patient may also begin utilizing this system of their own volition to manage their child's or their own respiratory illness. Regardless of the use case, the preferred embodiment as described below will be incorporated into a user's daily respiratory management routine.

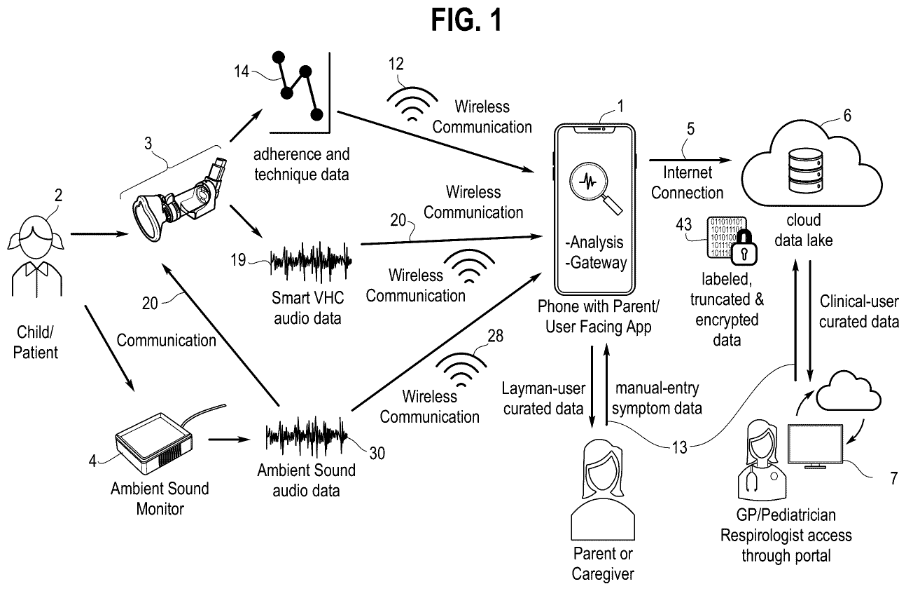

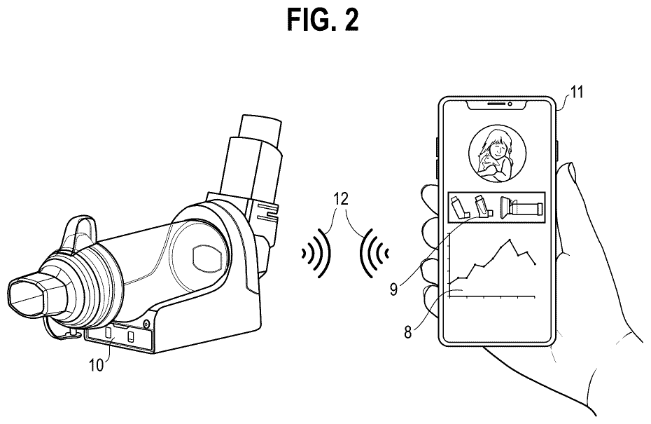

FIG. 1 illustrates one embodiment of a system illustrating how the devices may communicate, and how the different users (ex. child, parent, and physician) may interact with the system. The workings of this system will be detailed through the embodiment description. FIG. 2 shows one embodiment of a wireless communication between the Smart Valved Holding Chamber and the patient/caregiver application.

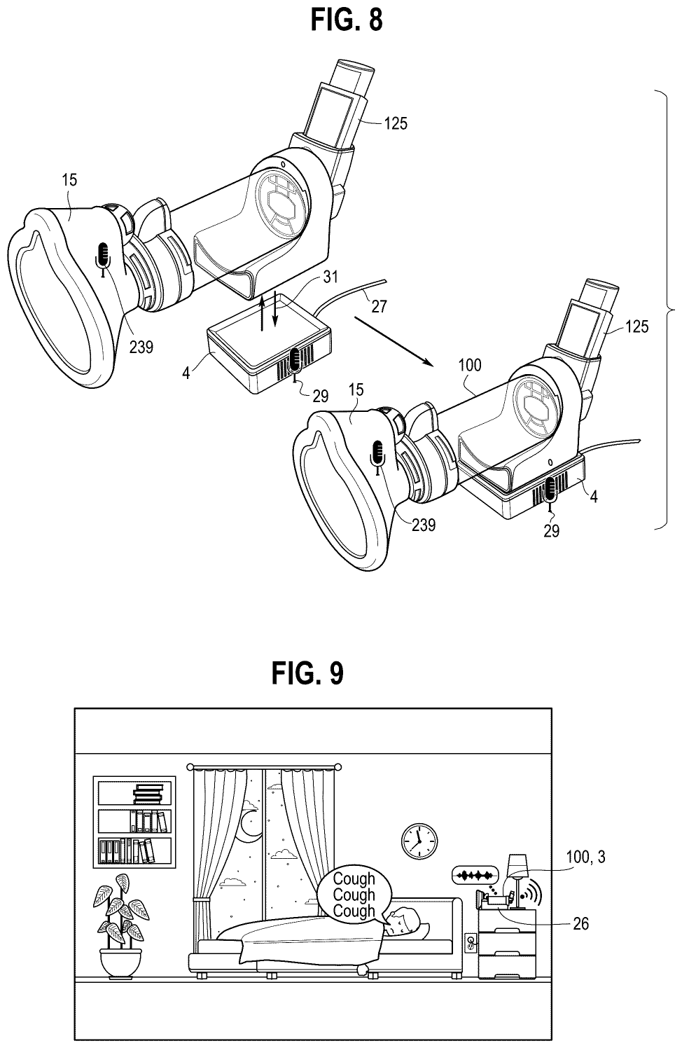

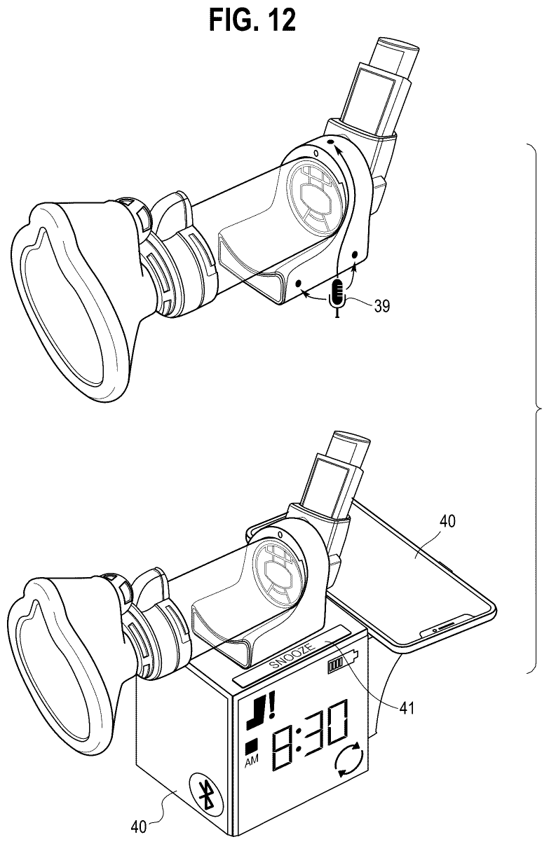

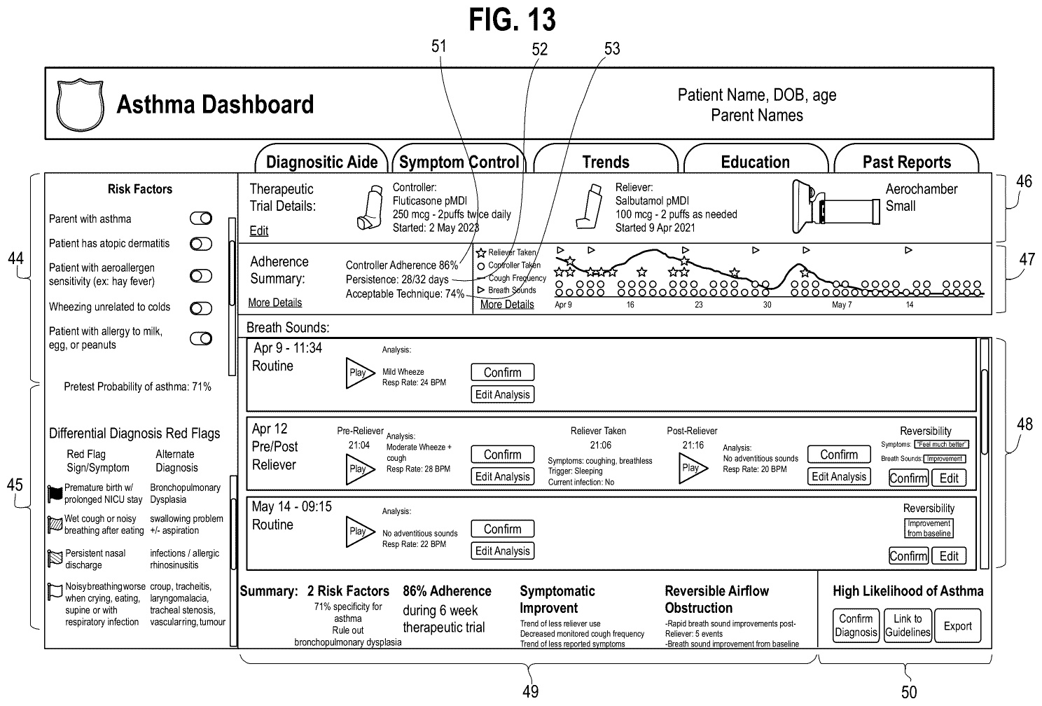

In one embodiment, the parent/patient facing app 1 conveniently stores the information regarding the management of the patient's respiratory condition. This application is connected to multiple data collecting devices that collect relevant information from the child 2 through the Smart Valved Holding Chamber 3 with beach sound recording and/or a separate Ambient Lung Sound Breath Recording device 4, as shown in FIGS. 8-12. This increased information can be used to assist in the management or diagnosis of the patient. The app may also connect to the internet 5 and upload the collected information to the cloud 6 where it may be accessed by a physician through a patient portal 7 to assist in the diagnosis of the disease or general management. Both the patient portal and parent/patient facing app include information that details the management of their child's symptom management 8 and medication regime 9.

To initialize the smart holding chamber 3, the battery 10 on the smart valved holding chamber 3 must be charged to a sufficient degree. If the battery is not charged the device will work in administering medication, but no information will be recorded for assistance in management. The smart valved holding chamber 38 is configured with a port providing for the battery to be charged via a charging cable (USB-C, etc.) or a wireless charging solution. With a charged device, the accompanying application must be downloaded and opened on a smart device 11 and connected to the smart valved holding chamber through a means of wireless communication 12. Upon connection, relevant information regarding the patient's disease, including prescription information, is to be entered either by the patient or the physician 13. Once the smart valved holding chamber is initialized, the device is ready for use.

The smart valved holding chamber of the described embodiment may include pressurized metered dose inhaler recognition 14, medication adherence tracking 14, inhalation technique scoring 14 which is calculated through available metrics like flow rate, device orientation, and other metrics, and wireless communication 12 with an accompanying application, among other features.

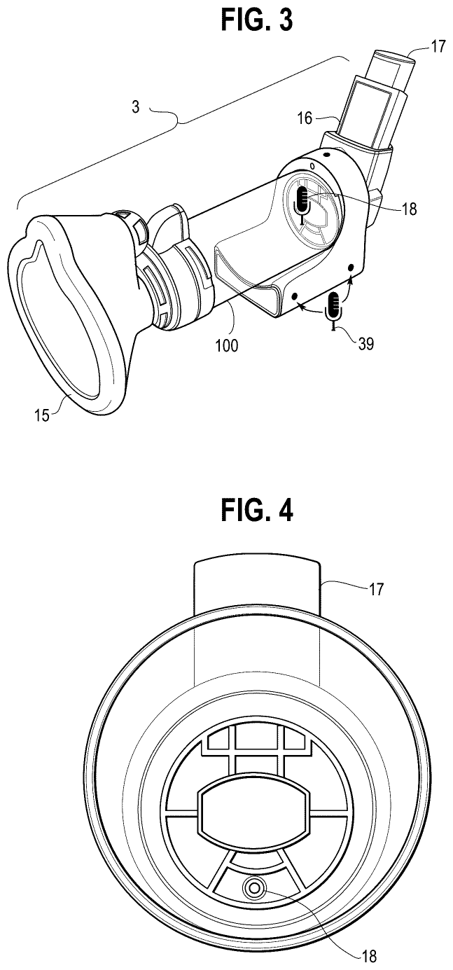

One embodiment can be seen in FIG. 3, which includes a user interface, configured for example and without limitation as a mouthpiece or mask. In one embodiment, the smart valved holding chamber may incorporate a mask 15 instead of a mouthpiece to fluidly connect to the airway of the patient. When in use, the mask is to be gently pushed onto the patients face to create a seal around the mouth and nose of a patient. This mask accommodates the use of the smart valved holding chamber 3 with young children incapable of using a mouthpiece.

The smart valved holding chamber 3 of the current embodiment is to be used whenever a user needs to take medication through a pressurized metered dose inhaler (pMDI). To take medication, a user inserts the desired inhaler into the rear of the smart valved holding chamber 3 and positions the mouthpiece in the mouth, or the mask onto the face. The user, patient and/or caregiver then depresses the top of the inhaler container 17 to release aerosolized medication into the device while the patient simultaneously inhales through the mask 15 or mouthpiece of the device. In one embodiment, the VHC 3 includes a feature that enables the recording of breath sounds while a patient is using the device. These breathing sounds will be automatically recorded through a microphone placed in a manner that will record sounds generated within the patient's lungs 18. Capturing this information requires that the microphone is positioned within the smart valved holding chamber such that it is either adjacent to, on, or within the body of the device to enable the capture of high-fidelity audio 19.

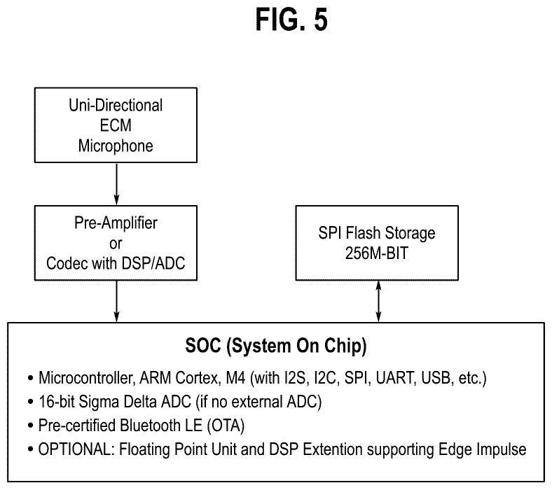

In one embodiment, the microphone 18 is positioned at the back of the VHC, and is attached to the MDI adapter as shown in FIG. 4. The acoustic-input of the microphone in this embodiment is thereby facing the back of the VHC valve. Alternate placements of the microphone 18 include in a port (i.e. a hole) located in the sidewall of the VHC body or in the large baffle at the valve seat as shown in FIG. 5. A protective, non-porous membrane will prevent the microphone from contamination while still permitting sound to reach the microphone regardless of the microphone's location. The microphone is preferably a unidirectional electret condenser microphone (ECM) whose orientation is optimized to pick up sound coming from the mouthpiece/mask adapter of the VHC. A MEMS microphone may also be used as an alternate microphone type but is not preferred. The ECM microphone will be more robust against potential contamination (from drug, condensation, water) as its diaphragm is larger than that of a MEMS microphone and there is also a protective felt cover. The unidirectionality of the microphone also makes it less prone to noise contamination. The microphone 18 is in communication with the interior of the holding chamber, including the interior of the cavity and/or user interface.

The microphone 18 signal will connect to a pre-amplifier before entering a 16-bit sigma-delta analog-to-digital converter (ADC) on the microcontroller. Alternatively, the microphone 18 may connect to an audio codec with sigma-delta ADC and gain control before being sent to the microcontroller as a digital signal through an 12C interface. In either case, the sound will be sampled at 16 kHz.

It may be desirable for the users to store the sound recordings onboard the VHC in the event the user's phone is not nearby to send the data to via Bluetooth. For this reason, the VHC architecture may include onboard flash storage proportional to the product of sampling rate (kHz), sample size (16-bit), sample duration (seconds), and number of samples to be stored. A serial peripheral interface (SPI) interface may be used for communication between the microcontroller and flash module. Additionally, an SD card module can be included for additional memory and data transfer capabilities. An architecture diagram of the microphone and supporting hardware can be seen in FIG. 5 (SD card not shown). The architecture may also include other systems such as power management or wireless communications transmission/receiving blocks. In one embodiment, a System on a Chip (SOC) will be used which will contain the microcontroller and other features to enable wireless communications, such as Bluetooth Low Energy (BLE).

To aid in this capture of audio data, one or more microphones are added to the device 39 to enable active noise cancellation of external sounds while the device is listening for breathing sounds through the microphone mentioned earlier 18. These additional microphones 39 may also be used for other purposes, like ambient noise monitoring for the detection of respiratory events occurring near the device. The data recorded will be temporarily stored on the device where it may be processed before it can be wirelessly transferred 20 to the application on the user's phone 1. The processing may include any steps that reduce the magnitude of data that needs to be transferred. Sound recognition algorithms may also run on the smart valved holding chamber.

If the user takes rescue medication (as opposed to controller medication) through the smart valved holding chamber, the patient may be prompted to repeat the same procedure a short period of time after the rescue medication was taken. This time, the device may be used without actuating the metered dose inhaler, therefore the user will not take any medication. Breathing through the valved holding chamber 3 will allow the onboard microphone that records sounds within the patient's lungs 18 to record respiratory related audio data. This recording will be used as a comparison against the original recording to enable physicians or caregiver to contrast differences in the patient's breath sounds before and after the rescue medication took effect. This data will be processed and stored in the same manner as described before.

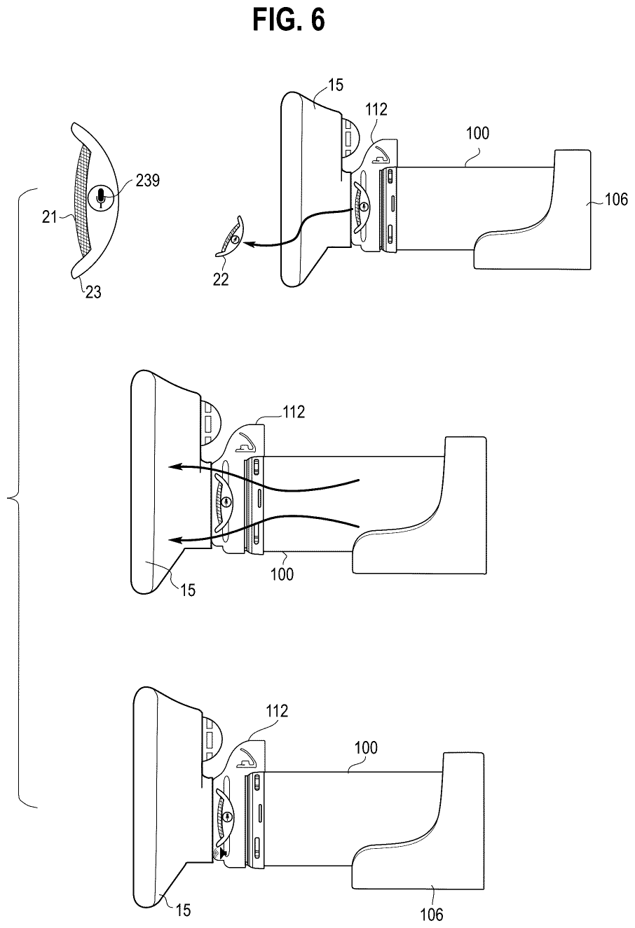

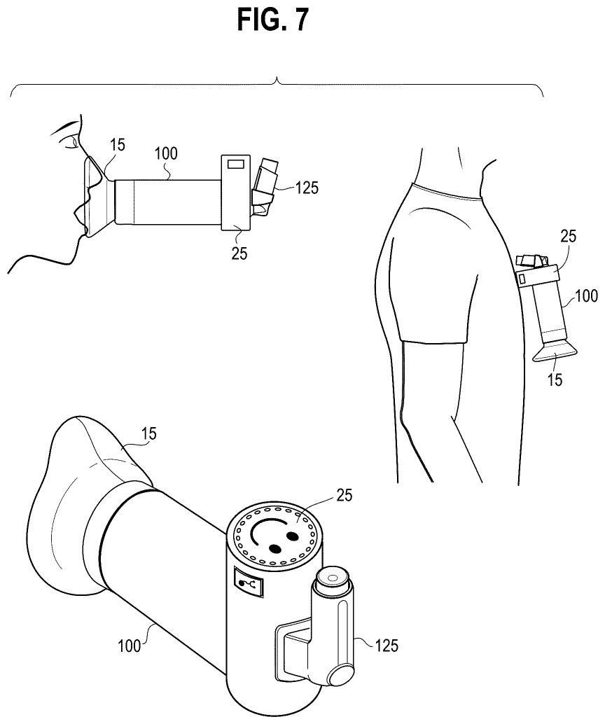

The smart valved holding chamber may have other features as seen in FIG. 6 and FIG. 7, including:
1. A microphone placed in the large baffle of the valve seat instead of in the backpiece on which a mesh cover, or protective element 21 either configurable or built into the microphone apparatus that minimizes the static or background noise sounds generated in the sound recording when air passes directly over or into the microphone.
2. A removable microphone element 22 to enable easier cleaning of the device, or ease in charging the element. The removable element may also facilitate the simple repair of the element or enable a user an option to not include breath sound recording if they feel it is an invasion of their privacy.
3. A cover, or protective element 23 that intrinsically repels suspended medication and particulate in the air and minimizes the chance of buildup on the recording device.
4. Integrated algorithms that reduce any static or constant noise generated through the rushing of air overtop of, or into the recording device.
5. A sound emitting device 24 placed alongside the microphone or recording element to enable periodic tests to assess the calibration of the microphone recording sounds over time. This would be worthwhile to ensure that the microphone does not become sufficiently clogged or dirty through everyday use. The sound emitting device may be a feature that is built into the VHC 3 or a feature that has another function that makes a consistent noise.
6. An electronic stethoscope 25 incorporated into the smart valved holding chamber instead of a microphone 18 to capture lung breath sounds. This stethoscope feature would be used by placing the device against the chest, back, or neck of the patient record breath sounds 25.
7. A GPS system built into the smart valved holding chamber. One potential application would be to record locations of the use of the device to capture more information and help users and physicians understand trends in usage.

Another element of this system may be the ambient sound monitoring device 4. The device is to be set up in an area where the patient resides for extended periods of time, e.g., most of the day/night, for example a bedroom 26. The device is configured by plugging it into electricity 27 and wirelessly pairing it with, or physically connecting it to either the smart valved holding chamber 3 or the accompanying app on the patient's smartphone 28. Once initialized, the device autonomously and actively listens through one or more microphones 29 for respiratory sounds like cough, wheeze, reparatory rate, or other lung related sounds that can be discerned from a distance. This information is recorded 30, processed and sent to either the smart valved holding chamber 20, or the patient app 28. In the present embodiment this device is separate from the smart valved holding chamber 3 and acts as a docking station to enable data transfer between the two devices 31. The docking embodiment also includes either direct electrical connections or a wireless charging pad that function to charge the Smart Valved Holding Chamber.

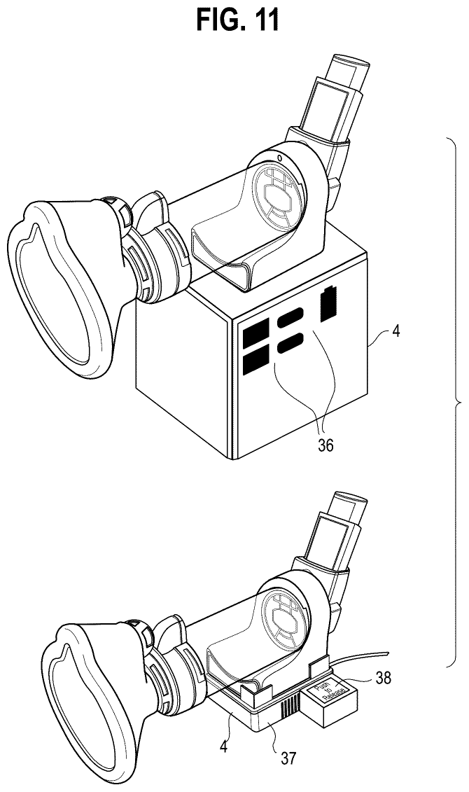

The ambient sound monitoring device may have other features as seen in FIGS. 10-12, including:
1. The external sensing device may also have either wireless 33 connectivity features to enable the transfer of the recorded data directly to the cloud 6, or other locations while bypassing the need to send information through the smart valved holding chamber 3 or the patient app 1.
2. In some embodiments this external device may also have LED lights 34 or an integrated display 35 to indicate the charging status of the smart valved holding chamber 3, a reminder for the user to take medication, to display that the process of data transfer is happening, a count for the number of times the device has picked up on a patient's respiratory sounds, or other notifications or information that can be presents to the user.
3. Other embodiments may include ports for connection 36 to other devices can be included like a USB port or other standardly widely recognized ports for data transfer, charging, or other uses.
4. Other elements of the embodiment include a locking mechanism 37 so that the smart valve holding chamber 3 is locked into place to prevent young children from tampering with the device while it is actively recording breath, cough, and other respiratory sound data. The locking mechanism may prevent children from accessing the inhaler pMDI medication, which may be conveniently left inserted into the VHC for quick administration in the event of an exacerbation. The locking mechanism can be unlocked by the push of a mechanical button 38 or other suitable locking elements. This locking mechanism could also be part of the VHC instead of the dock with ambient sound monitoring.
5. Other embodiments involve the described sensing apparatus being directly integrated into the smart valved holding chamber with microphones and wireless communication equipment built within, on, or adjacent to the smart valved holding chamber 39.
6. The docking embodiment may also include unrelated functionality to expand to different use cases, or to increase the overall function. These features include the addition of a Bluetooth enabled speaker system 40, an alarm function 41, wireless charging capabilities 42, or integration with smart home technologies/automation.
7. A specifically designed nighttime embodiment that includes a feature set for increased child monitoring. This embodiment enables continuous audio and/or video monitoring through a smartphone. Also, the smart valved holding chamber would have nighttime lighting that would glow to indicate the approximate time to the child to assist in sleep training. The glow would also assist in administering medication in the event of an exacerbation.
8. The ambient sound monitor, or dock, may have a button that would trigger the smart valved holding chamber to make noise and/or flash it lights to notify the user of its location. This is especially useful in situations where a child or user is experiencing an exacerbation and the patient, parent and/or caregiver is unable to locate the device to administer the medication.
9. The docking embodiment would facilitate the placement of a non-smart valved holding chamber, where upon placement or removal of the device the dock would alert the patient to update the child's conditions in the app, provide an approximate timestamp of when medication was taken, provide audiovisual instructions on how to use the device, or it could send out a periodic reminder to parents that the device is not on the dock, ensuring that the device's location is known at all times in the event of an exacerbation.

Data synchronization may be triggered manually or automatically when both the application and the smart valved holding chamber or ambient sound monitoring device is active and in close range.

On the app 1, there are analysis tools that depict the patient's medication adherence, inhalation technique, the number of times the ambient sound monitor picked up on coughs, wheeze, etc., and sound recordings from when the patient breathed through the smart valved holding chamber. The app also features a digital diary where a user can log symptoms related to their disease in order to identify patterns in their or their child's exacerbations. This information is displayed alongside external information brought in through the app including weather, pollen, air quality, and other location-based triggers pulled from the GPS of the smartphone or from the location of the devices that will help the user to better identify ways to avoid exacerbations. In addition, the app features an education section that teaches users various topics that will help them to better manage their condition. The app can also make use of the phone's camera function, and have a location to store photos, videos, or audio recordings taken by the parent or user.

The app also has a feature that enables the user to select information relavent to be shared with doctors. Information selected by users will be is formatted into a PDF that can be emailed or shared with physicians during visits. These PDFs can include embedded audio data, photos, or videos, that was recorded by the devices used in the system. Sharing this information with physicians enables them to no longer rely solely on the opinions of parents and make decisions based upon collected objective data. Additionally, the embedded/shared sound audio allows physicians to listen to a patient during an exacerbation (if the rescue medication was taken during an exacerbation), which would also help them in their decision making. Previously, the only way physicians could witness the child in an exacerbated state is if it happened during the physician visit, or if the parent managed to catch a video/sound recording to share it with the doctor. This audio data collected outside the smart valved holding chamber has varying levels of uncertainty as the information would be recorded with microphones that are not validated.

When connected to the internet, all the information stored on the application will also be uploaded to the cloud 5,6, where the information is encrypted 43 and securely stored for access by those who have permission, like the physician. This enables the physician to access the information of the patient without needing to wait for the patient or caregiver to create and send a PDF report.

The majority of data interpretation and processing will occur on the patient/caregiver application, or the cloud. Through an artificial intelligence or other preprogrammed algorithm, the recorded sounds will be analyzed for important markers that can be conveyed to caregivers, patients, or physicians. This data includes at least one of the following measures: presence of wheeze, breathing rate, identifying characteristics and trends of each breath/breathing pattern, classification of breathing patterns, etc. If processing occurs on the cloud, the information can be accessed by the patient or physician's information portal where it will be displayed for interpretation by the user. Otherwise, the information processed on the patient application will be sent to the cloud, where it can be accessed by the physician for review.

The physician accesses this information through a physician portal/application 7, which like the patient/guardian application, combines all the collected information from the described devices within the present disclosure. A representative image of the physician portal/application can be seen in FIG. 132. This application stores all the relevant information that enables the patient or the caregiver to review the collected data and make informed decisions concerning subsequent steps in diagnosis or management of the patient's condition.

The dashboard includes all of the information that the patient facing application includes, like risk factors 44, potential differential diagnoses 45, therapeutic trial details 46, an adherence summary 47 with a detailed breakdown of each breath sound each event 48, and an overall at-a-glance summary of the patient 49 with a recommendation for diagnosis 50 that must be confirmed by the physician. The adherence summary lists useful information to physicians, like the patient's overall controller adherence 51, persistence 52, and technique score 53. It also includes a chart displaying a visual a day-by-day history of the patient's trial information, allowing the physician to better discern trends in the patient's adherence to the therapeutic trial.

The physician portal also features integration with current Electronic Medical Record (EMR) software, which incorporates information about the patient directly into their workflow.

Although the present invention has been described with reference to preferred embodiments, those skilled in the art will recognize that changes may be made in form and detail without departing from the spirit and scope of the invention. As such, it is intended that the foregoing detailed description be regarded as illustrative rather than limiting and that it is the appended claims, including all equivalents thereof, which are intended to define the scope of the invention.

1. An asthma diagnosis and management system comprising:
a valved holding chamber defining an interior cavity, a backpiece having an opening configured to receive a pressurized metered dose inhaler and a user interface; and
a microphone coupled to the valved holding chamber and in communication with an interior of the valved holding chamber, wherein the microphone is configured to capture a sound of a user's lungs.

2. The asthma diagnosis and management system of claim 1 wherein the microphone is attached to the backpiece.

3. The asthma diagnosis and management system of claim 2 wherein the valved holding chamber comprises a valve assembly longitudinally spaced from the backpiece, wherein the microphone faces the valve assembly.

4. The asthma diagnosis and management system of claim 3 wherein the microphone comprises an ECM microphone.

5. The asthma diagnosis and management system of claim 1 wherein the valved holding chamber comprises a valve assembly longitudinally spaced from the backpiece, wherein the microphone is coupled to the valve assembly.

6. The asthma diagnosis and management system of claim 5 wherein the valve assembly comprises a baffle, wherein the microphone is coupled to the baffle.

7. The asthma diagnosis and management system of claim 1 wherein the microphone is releasably coupled to the valved holding chamber.

8. The asthma diagnosis and management system of claim 1 further comprising a protective element covering the microphone.

9. The asthma diagnosis and management system of claim 1 further comprising a sound emitting device coupled to the valved holding chamber and disposed adjacent the microphone, wherein the sound emitting device is configured to emit a sound for calibration of the microphone.

10. The asthma diagnosis and management system of claim 1 further comprising at least one secondary microphone coupled to the valved holding chamber.

11. The asthma diagnosis and management system of claim 10 wherein the secondary microphone is in communication with an exterior ambient environment of the valved holding chamber.

12. The asthma diagnosis and management system of claim 1 further comprising an ambient lung sound breath lung recording device comprising a microphone and configured to autonomously and actively record respiratory sounds, wherein the ambient lung sound breath lung recording device may be paired with the valved holding chamber.

13. An asthma diagnosis and management system comprising:
a valved holding chamber defining an interior cavity, a backpiece having an opening configured to receive a pressurized metered dose inhaler and a user interface; and
an electronic stethoscope coupled to the smart valved holding chamber and configured to capture a sound of a user's lungs.

14. A method of diagnosing and managing a user's asthma comprising:
actuating a pressurized metered dose inhaler coupled to a backpiece of a valved holding chamber;
inhaling through a user interface component of the valved holding chamber;
capturing a sound of the user's lungs while inhaling with a microphone in communication with an interior of the valved holding chamber;
waiting a predetermined period of time; and
breathing through the valved holding chamber without actuating the pressurized dose inhaler after the predetermined period of time and capturing a second sound of the user's lungs while breathing through the valved holding chamber.
